(19) United States
(12) Patent Application Publication
(10) Pub. No.: US 2018/0279879 A1
(43) Pub. Date: Oct. 4, 2018

(54) SYSTEM AND METHOD FOR OBTAINING AND WIRELESSLY TRANSMITTING ECG DATA FROM A PATIENT

(71) Applicant: John W Both, Malverne, NY (US)

(72) Inventor: John W Both, Malverne, NY (US)

(21) Appl. No.: 15/939,237

(22) Filed: Mar. 28, 2018

Related U.S. Application Data
(60) Provisional application No. 62/477,996, filed on Mar. 28, 2017.

(51) Int. Cl. A61B 5/00; H04L 29/08

(52) U.S. Cl. CPC A61B 5/0006; A61B 2560/0412; H04L 67/12

(57) ABSTRACT

An ECG patch has a waterproof housing, a microcontroller, one or more sensors, a storage medium, and a wireless transmitter; wherein the sensor captures signals from the body, stores the captured data on the storage medium, and transmits the captured data to a receiver using a wireless protocol. The receiver is connected to a server using a wireless protocol and transmits the captured data to the server for analysis and reporting.

### CROSS-REFERENCE TO RELATED APPLICATIONS

This application claims the benefit of U.S. Provisional Application Ser. No. 62/477,996 filed on Mar. 28, 2017, which is incorporated herein by reference.

### TECHNICAL FIELD

The present disclosure relates to medical devices and systems. More particularly, the present disclosure relates to a wireless sensor that is configured to perform an ECG on a patient and wirelessly transmit the ECG data to a medical professional for review.

### BACKGROUND

Heart disease is the leading cause of death among men and women both in the U.S. and worldwide. It's responsible for approximately 611,105 deaths each year and costs the U.S. $108.9 billion annually. Together, heart disease and stroke cost more than $312.6 billion in healthcare expenditures and lost productivity annually. The key to preventing death from heart disease is to protect the heart and know the warning signs and symptoms of a heart attack and stroke. Monitoring of the heart by utilizing traditional Holter and Event monitoring tests have been extremely effective in detecting abnormalities or arrhythmias in the heart that lead to heart attack and stroke. Traditionally, such tests were performed by devices that were strapped to a patient's belt with up to 5 leads attached to the chest and worn for 24 hours or up to 30 days. These devices are cumbersome, not waterproof, have a low patient compliance, rely on old technology, and do not monitor or report in real time. These devices required patients to remove and replace the leads, which often causes distortion and/or the loss of critical data. Example technology includes in the Philips Digitrak XT Holter.

Some new devices use wireless patch technology, but don't monitor, diagnose, or present data in real time to doctors. These patches store the data which must be retrieved in retrospect, often taking weeks for the doctor to receive a report after the test is complete. For example, the Zio Patch is a large unit that is worn on the upper chest for up to 14 days. It monitors the heart and stores ECG data in the unit. It cannot be worn in water and must be removed to shower. The unit is then mailed to the company, read, interpreted and a report is generated retrospectively and sent to the doctor. Other examples include CardioNet's MCOT, the LifeWatch MCT 3L, and the Preventise BodyGuardian®—none of which are waterproof and require the patient to remove and replace for showering or bathing. Such procedures not only reduce accuracy due to patient placement of the patch, but also fail to capture significant data when the patch is removed by the patient for any type of water activity.

Other patches have attempted to solve the waterproof issue, such as the Medicomp TelePatch, but such patches still have several shortcomings, including the lack of memory onboard the patch. If the patient is not within range of the receiving handset, the data is lost.

As such, despite the prior art's attempts, there still remains a need for a wireless patch for obtaining ECG data, that is capable of storing the data when not paired to a receiver, and that can transmit stored data and real-time data simultaneously when paired with a receiver. The present disclosure seeks to solve these and other problems.

### SUMMARY OF EXAMPLE EMBODIMENTS

In one embodiment, an ECG patch comprises a waterproof housing, a microcontroller, one or more sensors, a storage medium, and a wireless transmitter; wherein the sensor captures signals from the body, stores the captured data on the storage medium, and transmits the captured data to a receiver using a wireless protocol. In one embodiment, the receiver is connected to a server using a wireless protocol and transmits the captured data to the server for analysis and reporting.

In one embodiment, the receiver may process the data before transmitting to a server. In another embodiment, the receiver may receive, analyze, and report the data without need for the server.

In one embodiment, a method of reporting ECG data to a medical professional comprises placing a wireless ECG patch on a patient, the ECG patch configured to capture body signals, store the signals as data on a storage medium, and wirelessly transmit the data for review by a medical professional.

In one embodiment, the ECG patch comprises a microphone for recording the heartbeat of a user. The recorded audio may be synced with the visual data presented to the healthcare professional, allowing the healthcare professional to both see and hear the patient's heart. By reviewing the heart sound, heart rates, presence of arrhythmia, and respirations, a cardiologist is able to make a much more complete diagnosis while the patient is still at home or otherwise away from the physician.

### BRIEF DESCRIPTION OF THE DRAWINGS

FIG. 1 is a block diagram of an ECG patch;

FIG. 2 illustrates a system for obtaining and wirelessly transmitting ECG data from a patient;

FIG. 3 is a flowchart of the process of an ECG patch communicating with a receiver;

FIG. 4 is block diagram of a system for obtaining and wirelessly transmitting ECG data from a patient;

FIG. 5 is a flowchart of the web service; and

FIG. 6 illustrates the system data workflow.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there is a need for a patch that does not require a patient to remove and replace, that can effectively capture data, and that can wirelessly transmit data for analysis and review by a medical professional in real-time. As will be appreciated from the below disclosure, the ECG patch, system, and method described herein solve these needs and others.

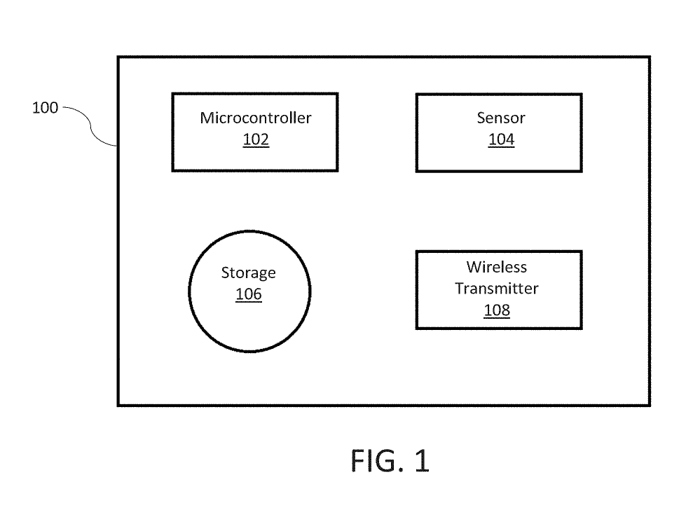

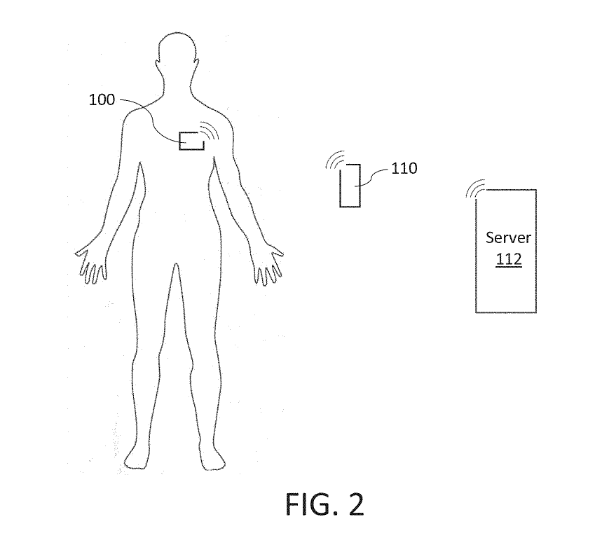

In one embodiment, as shown in FIG. 1, an ECG patch 100 comprises a microcontroller 102, one or more sensors 104, a storage medium 106 (e.g., non-volatile memory), and a wireless transmitter 108 (e.g., wireless network interface controller (WNIC) for Wi-Fi or Bluetooth®). As shown in FIG. 2, the ECG patch 100 captures signals from the body via the sensor 104, stores the captured data on the storage medium 106, and transmits the captured data to a receiver 110 (e.g., smartphone, tablet, computer, etc.) using the wireless protocol (e.g., Bluetooth®) of the wireless transmitter 108. In one embodiment, the receiver 110 is connected to a server 112 using a wireless protocol (e.g., Wi-Fi or Cellular) and transmits the captured data to the server 112 for analysis and reporting. Therefore, as shown in FIG. 2, for example, a patient would have at least one patch 100 placed on his/her body using adhesives known in the industry suitable for attaching to the body (e.g., hydrocolloid, silicone). The patch 100, powered by a battery (e.g., disposable zinc, although an alternative would be rechargeable, such as Li-ion), would then begin to capture ECG data using the sensor 104. The data is then temporarily held in flash memory or other storage 106 to await transmission to the receiver 110. If the patch 100 has a current Bluetooth® connection to the receiver 110, the data is transmitted from the memory/storage 106. If the patch 100 does not have a Bluetooth® connection, the data remains stored on the memory 106 until a connection is made. Once a connection is made and the data transmitted, the receiver 110 may then process and/or transmit the data to a server 112. Employing wireless protocols, memory on the patch 100, and waterproofing allows the patch 100 to remain on the patient constantly after the medical professional places the patch 100 in the desired location on the patient. This means that the patient will not need to remove the patch 100, ensuring that no data is missed. It also ensures proper placement of the patch 100, as the patient does not need to replace it. Further, because of the memory/storage 106 on the patch 100, losing a wireless connection (e.g., patient is sufficiently separated from a Bluetooth® or wireless signal that the patch 100 is not connected to any device) does not compromise data collection. These are all benefits over the prior art.

While a microcontroller is described above, it will be appreciated that other components may be used, such as a system on a chip (SoC).

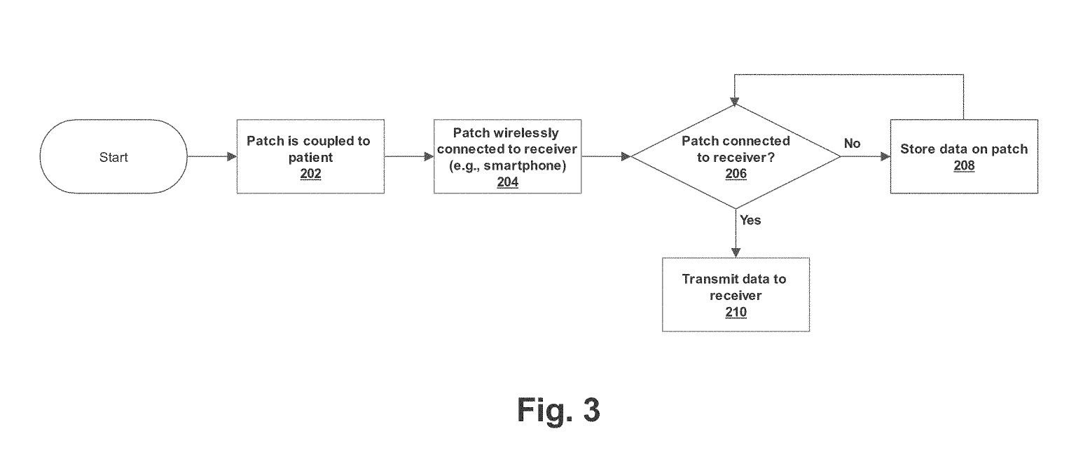

FIG. 3 illustrates a flowchart of the data process. In step 202, the patch is coupled to the patient. In step 204, the patch is wirelessly connected to a receiver. This may be accomplished using Bluetooth®, NFC, or other wireless technologies. The receiver, such as a smartphone, may comprise software for pairing with and collecting data. Such software may be downloaded and installed by the user or their healthcare provider. In step 206, the patch verifies a wireless connection to the receiver. If no connection is available, then in step 208 the data is stored on the patch. If the patch does have a connection, then the data is transmitted to the receiver. As appreciated, the patch 100 constantly verifies its wireless connection status to the receiver 110. In the event the patch and receiver are not in wireless communication with each other, the patch continuously stores the data in memory/storage. Upon connecting to the receiver once again, both the stored data and live data are simultaneously transmitted to the receiver 110. In one embodiment, the receiver 110 (e.g., the patient's smartphone) may process and transmit the data to a healthcare provider for review. For example, the processor in the smartphone may be utilized to analyze the data, generate a report, and then transmit the report (e.g., email, network, etc.) to the healthcare provider. As discussed earlier, and as shown in FIG. 2, an alternate embodiment utilizes a server 112, wherein the receiver 110 transmits the raw data to the server 112 for processing. Once processed, the server 112 would then transmit the data (e.g., in the form of a report) to the healthcare provider for review.

In one embodiment, the receiver may process the data and then transmit the processed data to a server. For example, a smartphone, using preprogrammed logic, could parse the data from the patch 100 using any number of parameters before transmitting to the server 112. This may be useful when hundreds of receivers are all transmitting to the server 112 at once. In such a scenario, the load of the server 112 may be lightened by using the receivers 110 to parse some of the data prior to transmission. In one non-limiting example, the mobile device (i.e., receiver) receives raw signal data. The mobile device separates the data into records, and the records may then be grouped into chunks. At this point, the chunks are transmitted to the server 112 where the chunks are grouped to services. The server may then execute parameters for sorting, removing duplicates, filter the data for particular regions of interest, and then present the data to a user/technician via a user interface (e.g., computer). The technician may then review, refine, and/or send a report based upon the data to the healthcare provider. In yet another embodiment, a server 112 may not be required. In other words, the receiver 110 may receive, analyze, and report the data without need for the server 112. The receiver 110 may then send the report directly to a medical professional via email or other communications protocol. In either scenario, the data may be processed according to user desires. For example, the data may be sorted, filtered, regions of interest selected based-upon preprogrammed parameters, etc. A report may be automatically generated, or the data presented to a technician via a user/healthcare provider interface (e.g., computer) where the user/healthcare provider may review the data and generate a report accordingly. The server may also couple information to the received signals based-upon the envelope of the signal. For example, the envelope may contain the MAC address of the patch that transmitted the data. The server may then use this MAC address to add client information (e.g., name, date of birth, etc.) to the data. In such a manner, if the data is intercepted between the patch, receiver, or server, no client information is compromised. However, other methods of securing such information during transmission may be used, such as using encryption or other known methods.

Because the data from the patch 100 is transmitted in real-time, a healthcare provider can be alerted the same day that a reportable event occurs (e.g., heart arrhythmia). This allows the healthcare provider to intervene to avoid more serious conditions (e.g., heart attack, stroke) that may present later on if not addressed. The healthcare provider may set the parameters for reportable events as well. To ensure accurate or additional readings, multiple patches 100 may be placed on a user, as selected by the healthcare provider. The patch 100 may comprise a housing that has been sealed so as to be waterproof, allowing a user to shower and perform other daily activities without removing the patch 100. Further, phone alerts may be configured to alert a user that the phone has lost its connection to the patch 100, allowing a user to take remedial action (e.g., step within range of the phone's wireless connection, or seek a new patch from the provider if the battery has died. In one embodiment, the battery may be rechargeable using contacts, magnets, or other components known in the art of waterproof rechargeable batteries systems. In one non-limiting example, the patch 100 may be used for a continuous six days. However, differing batteries will produce differing lengths of use. In one embodiment, the receiver 110 may display the current battery status of the patch 100.

It will be appreciated that the patch 100 may comprise sensors 104 of varying types. For example, the sensor 104 may comprise a breath detector, a temperature detector (e.g., thermistor), ECG electrodes, accelerometer, or others. Further, more than one sensor 104 may be present on the patch 100. For example, a patch 100 may comprise a both a breath detector and an ECG detector. Other variations, with more or less sensors 104 are contemplated. Using sensor(s) 104, biological data is then collected from the patient. It will be appreciated that while the examples herein contemplate using sensors and patches with ECG electrodes, such electrodes are not required. In other words, the patch 100 may collect other biological information (e.g., temperature, motion, respiration, etc.) for transmitting and does not require ECG data to be transmitted.

It will be appreciated that while the receiver 110 above was described as a smartphone, other devices capable of wireless communication may be used, such as smartwatches, tablets, or other known wireless communication devices.

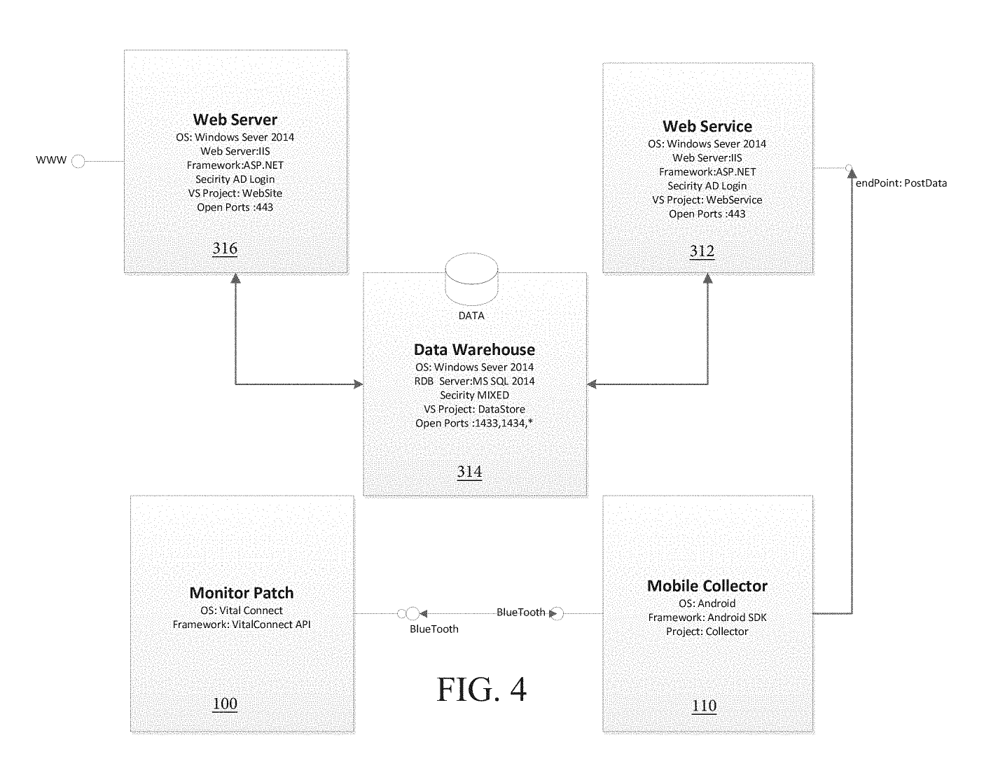

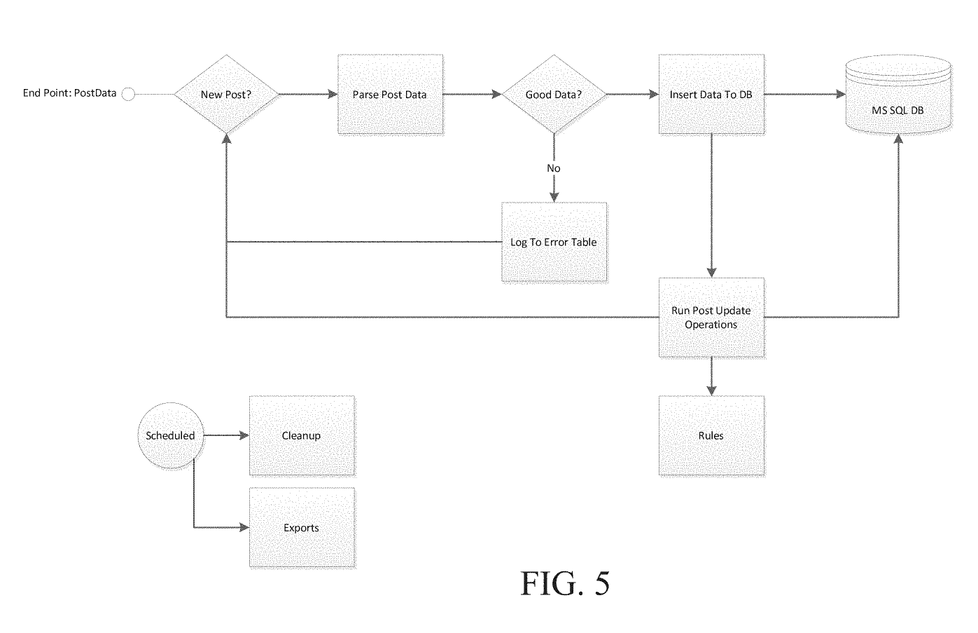

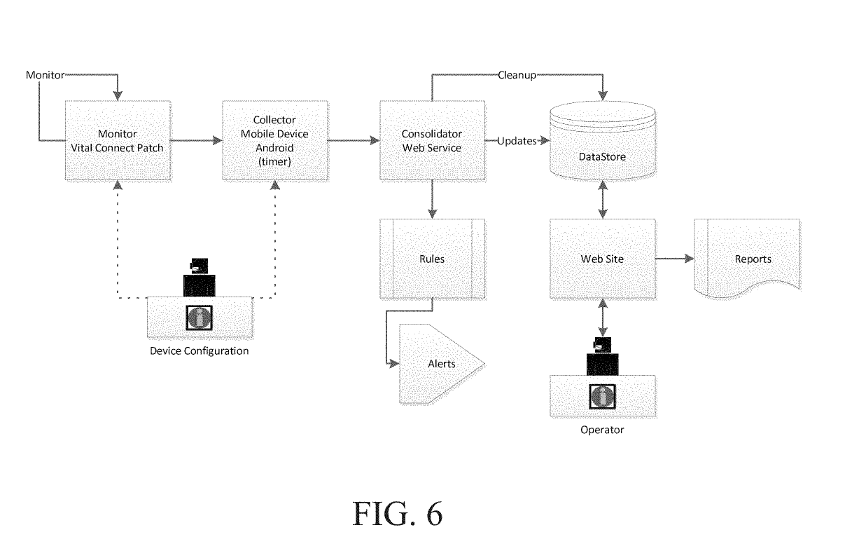

FIG. 4 illustrates an embodiment utilizing multiple services/servers. As shown, the patch 100 is connected via Bluetooth® to a mobile device. The data is collected by the mobile device and is posted to a web service. The data is then transferred and stored on a data warehouse, where it is accessible to a technician or other user via a web server. FIG. 5 illustrates a workflow of the web service 312. FIG. 6 illustrates a system data workflow for the data of FIG. 4.

In one embodiment, the ECG patch comprises a microphone for recording the heartbeat of a user. The recorded audio may be synced with the visual data presented to the healthcare professional, allowing the healthcare professional to both see and hear the patient's heart. By reviewing the heart sound, heart rates, presence of arrhythmia, and respirations, a cardiologist is able to make a much more complete diagnosis while the patient is still at home or otherwise away from the physician. Further, multiple patches may be placed on a user, allowing for more data gathering, and therefore, better assessment and use by a healthcare provider.

It is appreciated from the foregoing that the patch, system, and method for obtaining and transmitting ECG data solves various problems in the industry. In particular, because it is waterproof, a patient need not remove the patch for water activities (e.g., showering, exercising, recreation, etc.). This reduces lost data, as well as eliminates patient error when placing the patch. Further, due to the storage medium and wireless protocols employed, the data is able to be efficiently transmitted in real-time for review by a medical professional.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. An ECG patch comprising:
a waterproof housing;
a battery;
a microcontroller;
at least one ECG electrode; and
a wireless network interface controller.

2. The ECG patch of claim 1, further comprising non-volatile memory.

3. The ECG patch of claim 1, further comprising a temperature sensor.

4. The ECG patch of claim 1, further comprising an accelerometer.

5. The ECG patch of claim 1, wherein the wireless network interface controller is configured to transmit data to a receiver.

6. The ECG patch of claim 1, further comprising a microphone.

7. A method of using the ECG patch of claim 1 to transmit biological data from a patient to a healthcare provider, the method comprising:
a. adhering the ECG patch to the patient;
b. coupling the ECG patch to a receiver;
c. transmitting the biological data from the ECG patch to a receiver using the network interface controller;
d. transmitting the data from the receiver to a server; and
e. transmitting the data from the server to the healthcare provider.

8. The method of claim 7, wherein the biological data is processed by the receiver before being transmitted to the server.

9. The method of claim 8, wherein the receiver transmits the raw data to the server, wherein the server processes the data.

10. A system for continuously recording biological data from a patient and wirelessly transmitting it to a healthcare provider, the system comprising:
a biological patch comprising a waterproof housing, the housing containing one or more sensors, a battery, non-volatile memory, and a wireless network interface controller; and
a receiver, the receiver configured to communicate wirelessly with the biological patch;
wherein, when a wireless connection exists between the biological patch and the receiver, biological data is transmitted in real-time;
wherein, when a wireless connection does not exist between the biological patch and the receiver, biological data is stored on the non-volatile memory, and, upon obtaining a wireless connection, the stored biological data is transmitted simultaneously with the real-time biological data.

11. The system of claim 10, further comprising a server for receiving biological data from the receiver.

12. The system of claim 10, further comprising a healthcare provider interface.
